(19) United States
(12) Patent Application Publication
(10) Pub. No.: US 2002/0151973 A1
(43) Pub. Date: Oct. 17, 2002

(54) Intraocular lens

(75) Inventors: Tatsuo Arita, Kumamoto-shi (JP); Ichiro Sakurai, Yatsushiro-shi (JP); Noboru Komiya, Kawasaki-shi (JP)

Correspondence Address: ARMSTRONG,WESTERMAN & HATTORI, LLP, 1725 K STREET, NW., SUITE 1000, WASHINGTON, DC, 20006, US

(73) Assignee: Sakurai Seigi Co., Ltd., Yatsushiro-shi (JP)

(21) Appl. No.: 09/829,932

(22) Filed: Apr. 11, 2001

(51) Int. Cl. A61F 2/16

(52) U.S. Cl. 623/6.22; 623/6.13

(57) ABSTRACT

There is disclosed an intraocular lens which can easily obtain fine images of all objects in a range from a least distance of distinct vision to an infinity distance. The intraocular lens has: a lens unit including a convex lens, a hard magnetic material provided at the periphery portion of the convex lens, and a guidance member provided at the periphery portion of the lens unit; a cylinder-shaped flame unit for storing the lens unit therein, including a helical guidance groove formed on the inner surface thereof for meshing with a top of the guidance member of the lens unit and for carrying the lens unit along with the optical axis of the lens unit while rotating; and a pair of loop units for fixing the a cylinder-shaped flame unit storing the intraocular lens. The lens unit rotates by an application of magnetic force from outside thereby to move to a portion where the fine image formation of the desired object can be obtained.

### BACKGROUND OF THE INVENTION

The present invention relates to an intraocular lens used instead of a crystalline lens removed by cataract surgery.

A structure of an eyeball of a human is shown in FIG. 8. A cornea 81 is a transparent membrane placed at the forefront of the eyeball. There is placed an aqueous humor 82 in a gap between the cornea 81 and a crystalline lens 84. An iris 83 is a disc-shaped membrane with a circle-shaped small hole or a pupil placed at the center thereof, and it changes a diameter of the pupil according to a change in intensity of lights from outside or others. The diameter of the pupil changes in a range of about 2 to 5 mm. The crystalline lens 84 is a biconvex lens-shaped organ placed at the front portion of the eyeball. The crystalline lens 84 changes a thickness thereof in a range of about 3.7 to 4.4 mm, substantially changes a curvature of the front plane (on the cornea-side), thereby adjusting a focal distance. A ciliary body 85 drives a ciliary muscle for adjusting the crystalline lens 84. An image of a subject on the outside is formed on a retina 88. The lights received by the retina 88 are converted into electrical signals then transmitted to a cerebrum via an optic nerve 89. A sclera 86 protects the eyeball. A vitreous body 87 which occupies almost of the eyeball is made of a transparent sol-like material with a refractive index of 1.334 that is same to a refractive index of water.

The crystalline lens is an organ which plays the most important role of the eye. For a patient of cataract who has an optical disorder caused by clouding of the crystalline lens, the only effective choice for recovering his eyesight is a removal of such crystalline lens. In a state of so-called artificial absence of crystalline lens in which the crystalline lens being removed, the eye is very far-sighted since an image of the outside world can not be formed on the retina.

Therefore, to recover a function of eyesight nearly to that in a normal state, there is spreading a method of planting, instead of the removed crystalline lens, an artificial lens or an intraocular lens.

An example of an eyeball with an intraocular lens mounted therein is shown in FIG. 9. An intraocular lens 100 is made of, for example, a polymer such as polymethylmetacrylate-A (PMMA) and silicon, and fixed at a hole 91 for mounting a loop 92 provided at a predetermined position in the eyeball.

Here, since it is difficult to obtain fine images of the all objects in a range from at a least distance of distinct vision to an infinity distance if a focal distance is fixed, there is proposed an intraocular lens which can adjusting the focal distance.

In Japanese Unexamined Patent Publications Hei 9-294754 and Hei 11-276509, there are proposed, by the inventors of the present invention et al., methods of verifying a focal distance of an intraocular lens by changing a curvature of a spherical surface of an artificial lens, respectively. The intraocular lens proposed in the above-mentioned gazettes will be explained referred to as FIG. 10a and FIG. 10b. At a position where the crystalline lens had been provided originally, an intraocular lens 103 is fixed via a pair of loops 107 for fixing the intraocular lens 103 as shown FIG. 10a. The intraocular lens 103 is so-called a biconvex lens, and one plane thereof is made of a soft flexible film 106 and another a hard film 109. On the flexible film 106, there is formed a protrusion-portion 104 consisting of plural circle-shaped protrusions concentric with each other, excepting a circular region of 5 mm in diameter with a center of an optical axis of the lens 103. An internal space of the intraocular lens 103 and a storing portion 105 are connected with each other and filled up with a liquid or viscoelastic fluid. A magnetic member 108 glued to the top of the storing portion 105 is made of a soft or hard magnetic material. By an outer magnetic power, the magnetic member 108 moves in the horizontal direction in the figure. When the magnetic member 108 moves right in the figure, the liquid or viscoelastic fluid in the storing portion 105 moves to the internal space of the intraocular lens 103. Therefore, the protrusion-portion 104 is changed in shape thereby a curvature radius of the flexible film 106 being changed. By varying a radius of curvature of the intraocular lens 103 in such manner, the focal distance of the intraocular lens 103 is changed. Similarly, if the magnetic member 108 moves left in the figure, the liquid or viscoelastic fluid in the internal space of the intraocular lens 103 moves to the storing portion 105. That is, according to this gazette, the focal distance of the intraocular lens is controlled by applying a magnetic force from outside. In the gazette, as a means for forming a magnetic field, a glasses-shaped magnetic field-generation apparatus having solenoid coils is also proposed.

According to the above-mentioned gazette, there is an anxiety that electromagnetic waves influence to a user since the method requires a formation of the magnetic field at all times for verifying or maintaining a shape of the intraocular lens 103. To solve this problem, there is proposed an improvement of the intraocular lens of the above-mentioned gazette for avoiding a long term-exposure of the electromagnetic waves in Japanese Unexamined Patent Publication Hei 11-276509 by the inventors of the present invention et al. According to this gazette, there is provided a valve for controlling a flow of the liquid or viscoelastic fluid between the internal space of the intraocular lens 103 and the storing portion 105 thereby not requiring all times-supply of electricity.

However, a fine control of focal distance is difficult according to these methods. That is, a image of object at a least distance of distinct vision or an infinity distance can be formed on the retina easily, but a fine control is hard for forming images of the objects at a middle range in distance of about 3 to 10 m.

In Japanese Unexamined Patent Publication Hei 7-124185, there is proposed, by the inventors of the present invention et al., another method of enabling formations of images of objects in a range from a least distance of distinct vision to an infinity distance on the retina. In this method, an intraocular lens with a hard magnetic material provided on the periphery portion of the lens is pressed toward the retina by leaf springs while being drawn in the reverse direction by a magnetic force from outside. However, according to this method, a fine control is also hard for forming images of the objects at a middle range in distance of about 3 to 10 m since the intraocular lens is driven toward or against the retina in the vitreous body high in viscosity. Further, a response is slow since the lens is driven in the vitreous body of a sol-like material. In addition, there is an anxiety that electromagnetic waves influence to a user since the method requires a formation of magnetic field at all times by using a coil for verifying or maintaining the shape of the intraocular lens.

### SUMMARY OF THE INVENTION

The object of the present invention is to solve the above-mentioned problems thereby to provide an intraocular lens which can easily obtain fine images of all objects in a range from a least distance of distinct vision to an infinity distance.

An intraocular lens of the present invention has:

a lens unit including a convex lens, a hard magnetic material provided at the periphery portion of the convex lens, and a guidance member provided at the periphery portion of the lens unit;

a cylinder-shaped flame unit for storing the lens unit therein, including a helical guidance groove formed on the inner surface thereof for meshing with a top of the guidance member of the lens unit and for carrying the lens unit in the optical direction of the lens unit while rotating; and

a pair of loop units for fixing the flame unit storing the intraocular lens.

The convex lens and the guidance member are unified and are made of polymethylmetacrylate-A or silicon, for example. The hard magnetic material is in a shape of a film, for example.

The diameter of the convex lens is preferably set to 5 mm that is same to the maximum diameter of a pupil. For example, plate-shaped protrusions are provided at two portions on the periphery of the convex lens so as to be in symmetry with each other with respect to an optical axis of the convex lens, and a pair of guidance grooves are provided so as to be in symmetry with each other with respect to a center axis of the flame unit. Threads having a shape corresponding to that of the guidance grooves may also be provided at the periphery of the lens unit. The bottom of the guidance groove and the tip of the guidance member are both preferably processed into curved surfaces.

In the intraocular lens of the present invention, there is employed sole ring-shaped hard magnetic material provided so as to surround the convex lens. Similarly, plural arc-shaped hard magnetic materials may also be provided so as to surround the convex lens. For example, an even number of the arc-shaped hard magnetic materials magnetized in radial directions of the lens unit and those magnetized in the reverse directions are provided so as to adjoin to ones having a magnetization in the reverse directions.

A 2 mm-transfer of the lens unit in the flame unit along with the optical axis enables an adjustment of a focal distance from a least distance of distinct vision to an infinity distance.

### BRIEF DESCRIPTION OF THE DRAWINGS

FIG. 1 is a plan view showing an intraocular lens in one embodiment of the present invention.

FIG. 2 is a longitudinal cross sectional view showing a lens flame of the same intraocular lens.

FIG. 3a is a plan view showing a lens unit of the same intraocular lens, and

FIG. 3b is a side view showing the same.

FIG. 4a is a longitudinal cross sectional view showing the same intraocular lens, and

FIG. 4b is a vertically cross-sectional view showing a principal portion of the same.

FIG. 5a is a plan view showing a direction of magnetization of a hard magnetic material film formed on the lens unit of the same intraocular lens, and

FIG. 5b is a front view showing a person with the same intraocular lens mounted in his eyeball.

FIG. 6 is a plan view showing a lens unit used in an intraocular lens in another embodiment of the present invention.

FIG. 7 is a perspective view showing a lens unit used in an intraocular lens in still another embodiment of the present invention.

FIG. 8 is a longitudinal cross sectional view showing a structure of an eyeball.

FIG. 9 is a longitudinal cross sectional view showing an eyeball with an ordinary intraocular lens mounted therein.

FIG. 10a is a longitudinal cross sectional view showing an eyeball with an intraocular lens of comparative example mounted therein, and

FIG. 10b is a longitudinal cross sectional view showing a principal portion of the same intraocular lens.

### DESCRIPTION OF THE PREFERRED EMBODIMENTS

Hereafter preferred embodiments of the present invention would be described in detail referred to as the attached drawings.

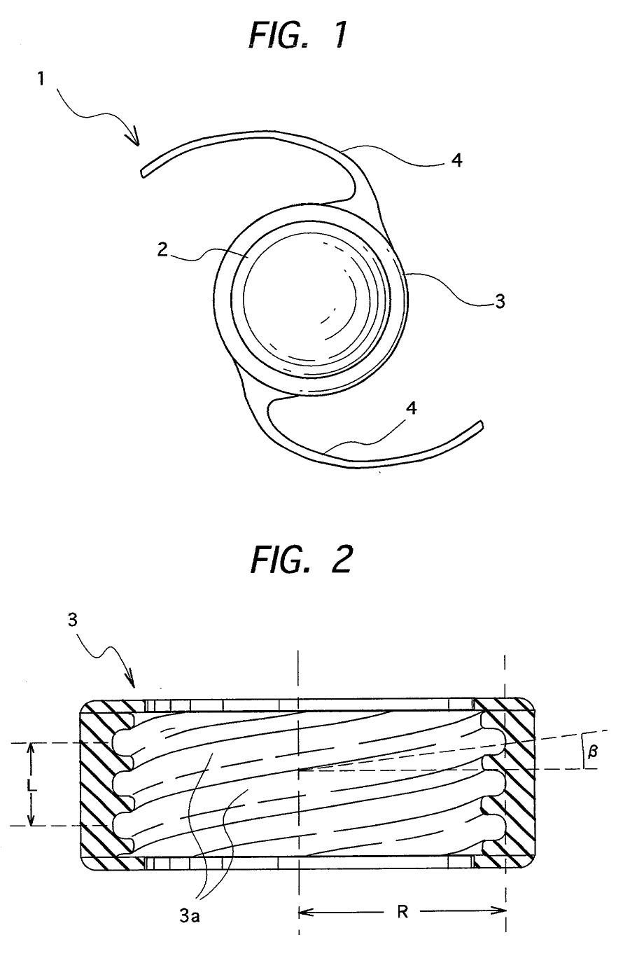

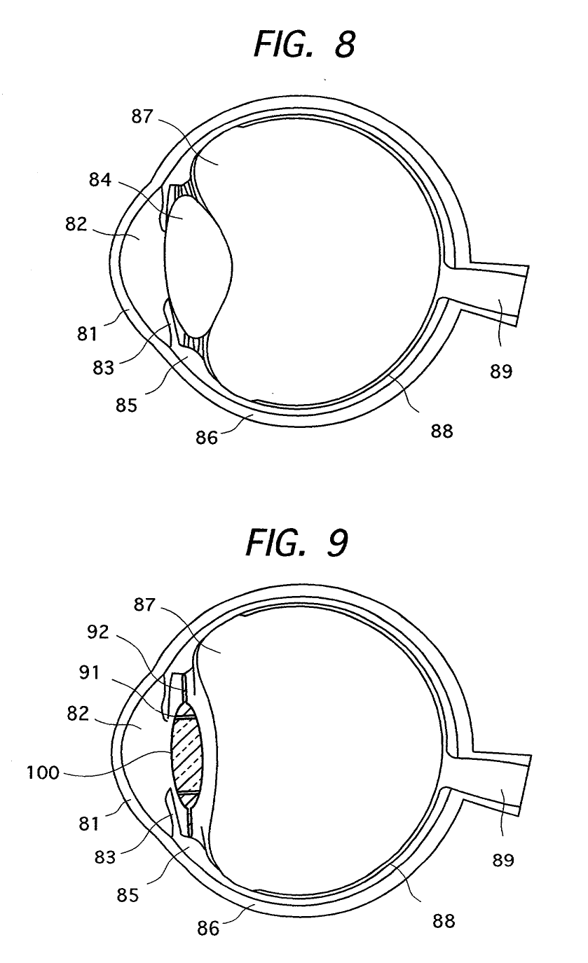

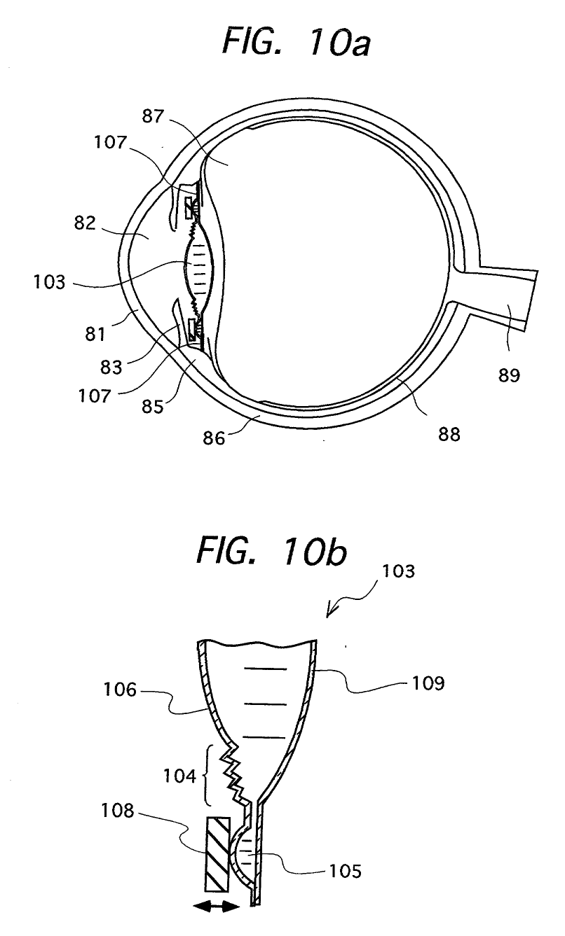

An intraocular lens of this embodiment is shown in FIG. 1. A lens unit 2 which is a substitution for a crystalline lens 84 in FIG. 8 is stored inside a cylindrical lens flame 3 made of a nonmagnetic material. To the lens flame 3 are attached a pair of loops 4 for fixing an intraocular lens 1 in a predetermined position in an eyeball.

The lens flame 3 has an inner thread-like shape with a pair of helical guidance grooves 3a with a lead angle of “β”. The pair of guidance grooves 3a are provided so as to be in symmetry with each other with respect to a center axis of the lens flame 3 represented by an alternate long and short dash line in the figure. The bottoms of the guidance grooves 3a are processed to have curved surfaces.

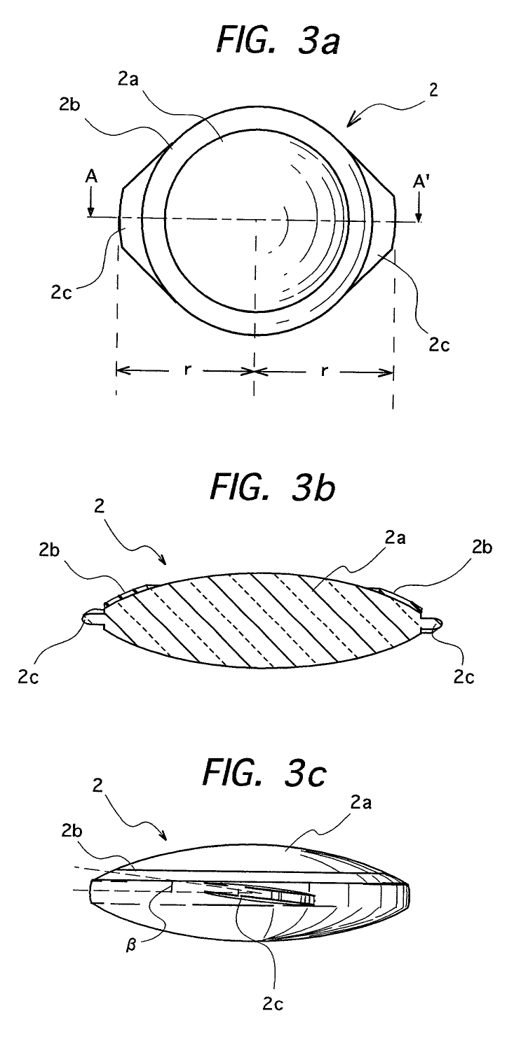

The lens unit 2 stored in the lens flame 3 is made of, for example, polymethylmetacrylate-A (PMMA), and has a biconvex lens-shaped lens portion 2a of 5 mm in diameter formed on the center thereof. The perimeter of the lens portion 2a to be placed on the cornea side is coated with a ring-shaped hard magnetic material film 2b excepting a pair of protrusion portions 2c. The protrusion portions 2c are provided at the periphery of the lens unit 2 so as to be symmetrical with each other with respect to the optical axis of the lens portion 2a. The protrusion portion 2c has an arc-shaped tip with a radius of “r” and a semicircular section, as shown in FIG. 3b. Here, the radius “r” is slightly smaller than “R” shown in FIG. 2 or a distance from the center axis of the lens flame 3 to the bottoms of the guidance grooves 3a. A principal place of the protrusion portion 2c is inclined with a predetermined angle of “β” with respect to the plane perpendicular to the optical axis of the lens unit 2. That is, the principal plane of the protrusion portion 2c has the same inclination with the lead angle of the guidance groove 3a represented by “β” in FIG. 2.

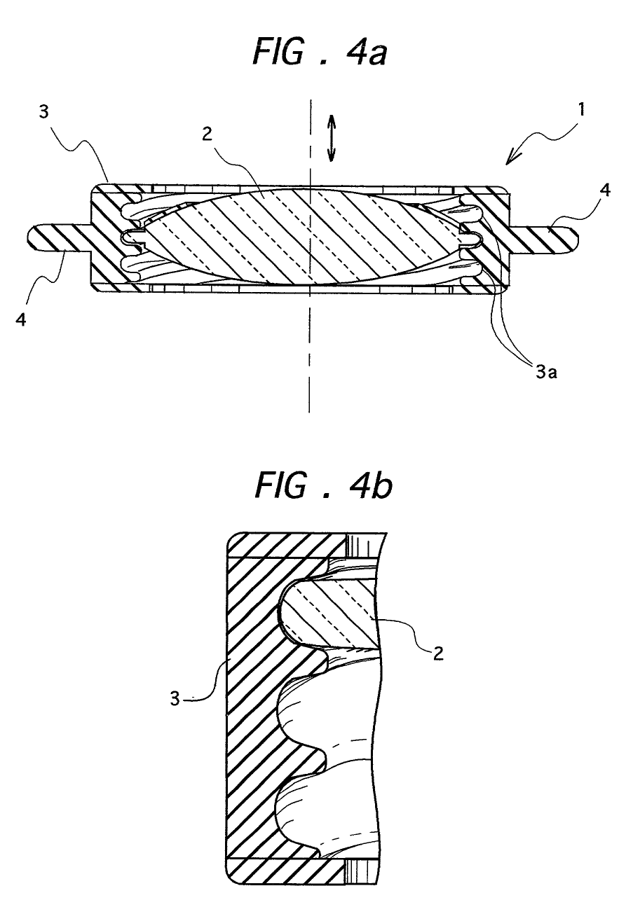

The lens unit 2 is stored inside the lens flame 3, with the pair of the protrusion portions 2c each being meshed with the guidance groove 3a as shown in FIG. 4a and FIG. 4b. The lens unit 2 and the lens flame 3 are loosely meshed with each other, and the lens unit 2 is provided in the internal space of the lens flame 3 while being allowed a rotation. The optical axis of the lens unit 2 and the center axis of the lens flame 3 are same, and the lens unit 2 can move only in the direction of the optical axis thereof represented by an arrow in the figure while rotating.

For example, a lead “L” shown in FIG. 2 is set to 2 mm, and the lens unit 2 is set to rotate one round in maximum inside the lens flame 3. In this manner, it becomes possible to form images of the objects at a range from a least distance of distinct vision to an infinity distance on the retina. The intraocular lens is set in the eyeball so as to obtain an image of the object at the least distance of distinct vision (the nearest object) when the lens unit 2 reaches one edge and to obtain that at the infinity distance when the lens unit 2 reaches another edge.

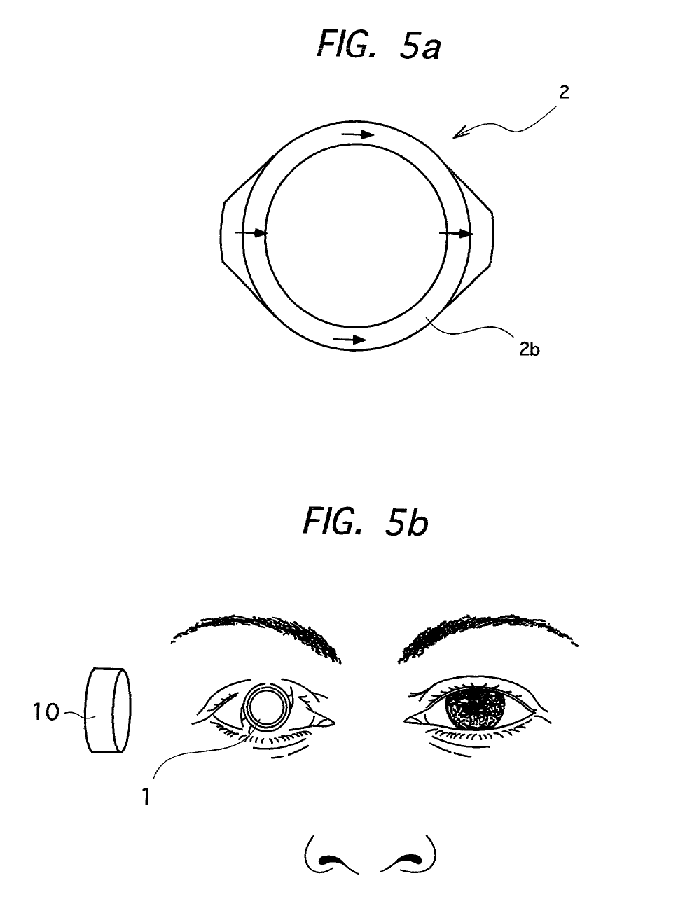

The intraocular lens I mounted in the eyeball rotates by an application of magnetic force from outside. The hard magnetic material film 2b is magnetized as shown in FIG. 5a, for example. When a permanent magnet 10 is provided at a temple of the user with the intraocular lens 1 mounted therein and the magnet 10 is moved, the lens unit 2 moves inside the lens flame along with the optical axis thereof while rotating by a force of attraction or that of repulsion functions between the hard magnetic material film 2b and the permanent magnet 10. The permanent magnet 10 is, for example, provided on an arm of glasses.

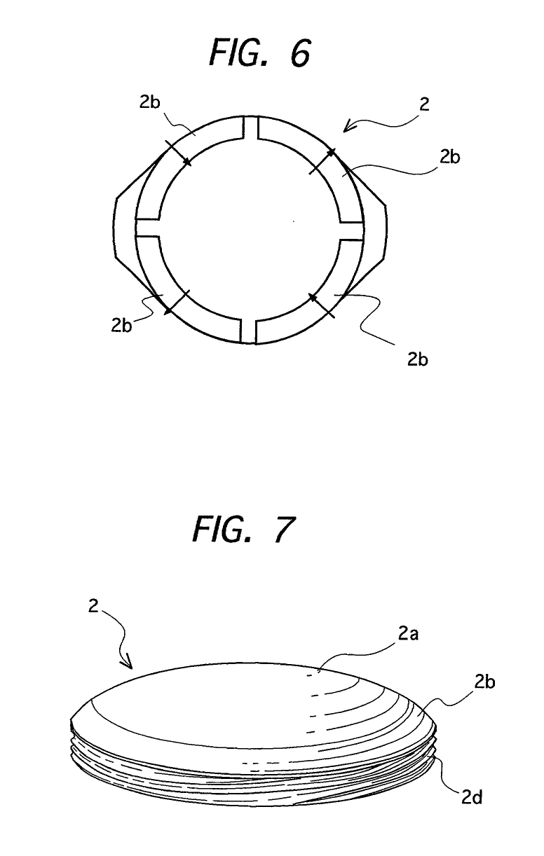

Plural number of the hard magnetic material members may also be provided on the surrounding of the lens portion. As shown in FIG. 6, a combination of hard magnetic material films 2a having a polarity different form that of the adjoining films 2a may also be employed, for example.

Further, there may also be employed a lens unit 3 having threads 2d of which shape is corresponding to that of the guidance grooves 3a as the above-mentioned protrusion portion as shown in FIG. 7. Although the lens unit 2 shows a larger friction resistance at move than the above-mentioned lens unit, it can move more stably inside the lens flame.

In the case of driving the intraocular lens as proposed in Japanese Unexamined Patent Publication No. Hei 7-124185, it is difficult to control a small distance of a transfer directly. On the other hand, according to the present invention, the distance of a transfer can be accurately controlled by control the rotation of the lens unit. Therefore, an easy control of a focal distance can be realized. A coil may also be employed instead of the magnet for driving the lens unit. In such case, an exposure of electromagnetic waves in all times as in Japanese Unexamined Patent Publication No. Hei 9-294754 can be avoided since an application of magnetic force from outside is required only at changing the focal distance.

What is claimed is:

1. An intraocular lens comprising:
a lens unit including a convex lens, a hard magnetic material provided at the periphery portion of said convex lens, and a guidance member provided at the periphery portion of the lens unit;
a cylinder-shaped flame unit for storing said lens unit therein, including a helical guidance groove formed on the inner surface thereof for meshing with a top of said guidance member of said lens unit and for carrying said lens unit along with an optical axis of said lens unit while rotating; and
a pair of loop units for fixing said cylinder-shaped flame unit storing said intraocular lens.

2. The intraocular lens in accordance with claim 1, wherein said guidance member includes a pair of plate-shaped protrusions provided at portions which are symmetrical with each other with respect to an optical axis of said convex lens, and a pair of said guidance grooves are provided in symmetry with each other with respect to an center axis of said flame unit

3. The intraocular lens in accordance with claim 1, wherein both a bottom of said guidance groove and- tip of said guidance member have curved surfaces.

4. The intraocular lens in accordance with claim 1, wherein said hard magnetic material is shaped into a ring surrounding said convex lens.

5. The intraocular lens in accordance with claim 1, wherein said hard magnetic material is in a shape of film.

6. The intraocular lens in accordance with claim 1, wherein said hard magnetic material is consisted of plural arc-shaped members provided to surround said convex lens.

7. The intraocular lens in accordance with claim 1, wherein a periphery portion of said convex lens on one surface of said lens unit excepting said guidance member is coated with said hard magnetic material.

8. The intraocular lens in accordance with claim 1, wherein said convex lens is made of polymethylmetacrylate-A or silicon.

9. The intraocular lens in accordance with claim 1, wherein both said loop member and said flame unit are made of nonmagnetic materials

10. The intraocular lens in accordance with claim 1, wherein said flame unit allows a transfer for 2 mm of said lens unit in a direction parallel to an optical axis of said lens unit.

11. The intraocular lens in accordance with claim 1, wherein said convex lens has a diameter of 5 mm.
